(19) United States
(12) Patent Application Publication
(10) Pub. No.: US 2025/0352657 A1
(43) Pub. Date: Nov. 20, 2025

(54) COMPOSITION FOR PREVENTING AND/OR TREATING A PATHOLOGICAL DYSBIOSIS OF THE INTESTINAL MICROBIOTIA

(71) Applicant: PLL THERAPEUTICS, BOULIAC (FR)

(72) Inventor: Jean-Pascal ZAMBAUX, BOULIAC (FR)

(21) Appl. No.: 18/860,881

(22) PCT Filed: May 5, 2023
(86) PCT No.: PCT/EP2023/062035

§ 371 Date: Oct. 28, 2024

(30) Foreign Application Priority Data
May 10, 2022 (FR) FR2204409

(51) Int. Cl. A61K 47/64; A61K 31/19; A61K 31/201; A61K 31/202; A61K 31/575; A61P 1/00; A61P 31/04

(52) U.S. Cl. CPC A61K 47/6455; A61K 31/19; A61K 31/201; A61K 31/202; A61K 31/575; A61P 1/00; A61P 31/04

(57) ABSTRACT

To meet the needs of new therapies for preventing or treating pathological dysbiosis of the intestinal microbiota, in particular for treating or preventing neurodegenerative or intestinal diseases. The invention relates to the use of compositions comprising a plurality of specific molecules for treating a pathological dysbiosis of the intestinal microbiota. The pathological dysbiosis of the intestinal microbiota can be characterized by an excess of sulfide-producing bacteria and a deficit in butyrate-producing bacteria.

### TECHNICAL FIELD

The invention concerns the prevention and/or treatment of pathological dysbiosis of the intestinal microbiota. In particular, the invention relates to the use of compositions comprising multiple specific molecules for treating a pathological dysbiosis of the intestinal microbiota.

### PRIOR ART

The microbiota is the collection of microorganisms (bacteria, archaea, viruses and eukaryotes) that thrive in a specific environment.

The human body contains a multitude of microbiota, notably in the skin, mouth, vaginal cavity, throat, and digestive system.

The microbiota present in the digestive system, also known as the intestinal microbiota, is distributed from the stomach to the colon and represents between 35 and 50% of the volume of intestinal contents for a weight of over 1 kilogram.

The intestinal microbiota is home to a large proportion of the 10 trillion microorganisms that inhabit the human body.

Although discovered in the 19th century, it wasn't until the advent of high-throughput sequencing in the 2000s that scientists were able to identify all the genes in the intestinal microbiota. Characterizing all the microbial genomes found in the intestine has led to the discovery of over a thousand different species, the vast majority of them bacterial.

In-depth analysis of the intestinal microbiota has shown that, like a fingerprint, the intestinal microbiota is unique to each individual and depends on their environment and genetics.

Once formed, the composition of the microbiota remains relatively stable throughout an individual's life. Variations are generally less than 5% of the total microbiota.

As a result, pathogenic conditions in the intestinal microbiota are difficult to detect, as they originate from these particular species.

When a patient presents with a disease, an imbalance of pathogenic intestinal flora known as pathological dysbiosis can be found; the threshold of imbalance is difficult to establish, as are variations in genera and species unless there is a biomarker for the disease.

Thanks to the removal of technical barriers, analysis of the intestinal microbiota has revealed its major role both in maintaining the body's good health and in the onset of numerous diseases.

The intestinal microbiota plays an essential role in the body's functioning. The microorganisms that make it up have a wide field of action, from breaking down food to epithelial repair to metabolic regulation.

Nevertheless, through dysbiosis, that is to say, a change in the composition of the microbiota, the microbiota can be associated with derived or induced pathological disorders such as neurodegenerative and intestinal diseases and certain cancers.

Central nervous system-intestine interactions are a major factor in the development of certain pathologies. In Parkinson's disease, the link between pathological dysbiosis of the intestinal microbiota and the onset of motor disorders and neuroinflammation has been demonstrated.

Pathological dysbiosis of the intestinal microbiota has been demonstrated in other neurodegenerative diseases, notably in patients with amyotrophic lateral sclerosis.

In addition, it is now known that intestinal diseases, and more specifically inflammatory bowel disease, Crohn's disease, and food intolerance, notably gluten intolerance, are linked to an intestinal microbiota that is depleted in microorganisms, and therefore the presence of pathological dysbiosis.

Currently, the proposed solutions for preventing or combating dysbiosis consist essentially in administering probiotics and/or prebiotics, particular to treat intestinal diseases, but their efficacy is highly variable and depends on the patient's basic intestinal flora.

Another promising solution to pathological dysbiosis is fecal transplantation. However, the effect on intestinal microbiota requires at least 2 transplants per week to achieve a lasting effect.

Moreover, with regard to degenerative diseases specifically, these are often very hard on the patient. Some symptoms may be reduced, but such diseases are never completely cured and there is currently no satisfactory treatment. Many chemical agents have therapeutic properties but their toxicity, their lifetime, or their rapid elimination make them unsuitable for treating degenerative diseases.

Specifically, despite them being multifactorial, current treatments target just one of the causal factors of these diseases, and do not seek to act more generally with a systemic approach directly targeting the causes of the disease. For example, Lou Gehrig's disease, or amyotrophic lateral sclerosis, is linked to numerous factors, in particular to oxidative stress, mitochondrial dysfunction, neuroinflammation, excitotoxicity, oligodendrocyte dysfunction and degeneration, altered proteostasis, an altered DNA repair system, changes in nucleocytoplasmic RNA and in RNA related to transport proteins, defective axonal transport, and defective vesicular transport as well as pathological dysbiosis of the intestinal microbiota. Currently, the treatment recommended for amyotrophic lateral sclerosis is Riluzole® which acts only on the inhibition of glutamate release in order to combat excitotoxicity, and no action on the intestinal microbiota is considered. The same observation can be made for other degenerative diseases.

There is therefore a strong need for a solution to prevent or treat pathological dysbiosis of the intestinal microbiota, in particular to treat or prevent neurodegenerative or intestinal diseases, and to meet an essential demand for new therapies.

The aim of the invention is therefore to meet all of these needs and to overcome the disadvantages and limits of the prior art.

### SUMMARY OF THE INVENTION

To meet this objective, the invention proposes the use of particular compositions for preventing and/or treating pathological dysbiosis of the intestinal microbiota, and suitable for use in treating or preventing diseases associated with pathological dysbiosis of the intestinal microbiota, in particular neurodegenerative or intestinal diseases.

To this end, the invention relates to a composition comprising at least:
lactic acid, and/or a salt and/or ester and/or anhydride of lactic acid,
butyric acid, and/or a salt and/or ester and/or anhydride of butyric acid, and
propionic acid, and/or a salt and/or an ester and/or anhydride of propionic acid, for use in humans or animals in the prevention and/or treatment of pathological dysbiosis of the intestinal microbiota.

Advantageously, the composition according to the invention is capable of restoring the balance of the microbiota of an individual presenting a pathological dysbiosis of the intestinal microbiota.

Preferentially, the composition according to the invention is used to treat a pathological dysbiosis of the intestinal microbiota characterized, in the intestinal microbiota, by an excess of sulfide-producing bacteria and a deficit in butyrate-producing bacteria.

Said treatment according to the invention is thus preferentially characterized in the intestinal microbiota by an increase in the proportion of butyrate-producing bacteria and a decrease in the proportion of sulfide-producing bacteria.

Advantageously, the effect on butyrate-producing bacteria and sulfide-producing bacteria provides a synergistic action for treating pathological dysbiosis and in particular pathological dysbiosis associated with pathologies.

More particularly, the composition can be used in humans or animals for the prevention and/or treatment of at least one neurodegenerative disease and/or intestinal disease associated with pathological dysbiosis of the intestinal microbiota. According to a particularly suitable variant, the composition can be used in humans or animals in the prevention and/or treatment of at least one neurodegenerative disease and/or intestinal disease associated with a pathological dysbiosis of the intestinal microbiota wherein the human or animal presents an intestinal dysbiosis characterized in the intestinal microbiota by an increase in the proportion of sulfide-producing bacteria and a decrease in the proportion of butyrate-producing bacteria.

Surprisingly, the inventors discovered that the composition according to the invention could treat or prevent diseases associated with pathological dysbiosis, and in particular neurodegenerative diseases or intestinal diseases associated with pathological dysbiosis such as lateral sclerosis or intestinal diseases such as Crohn's disease.

The composition according to the invention can be used as a drug or dietary supplement for humans or animals.

According to one variant, the composition according to the invention can be used to prevent or treat a neurodegenerative disease associated with a pathological dysbiosis of the intestinal microbiota selected from amyotrophic lateral sclerosis, multiple sclerosis, Parkinson's disease and Alzheimer's disease.

According to another variant, the composition according to the invention can be used to prevent or treat an intestinal disease associated with pathological dysbiosis of the intestinal microbiota selected from Crohn's disease, chronic inflammatory bowel diseases, hemorrhagic rectocolitis, irritable bowel syndrome, ulcerative colitis, rheumatoid arthritis and food intolerance, in particular gluten intolerance.

According to a particularly suitable embodiment, the composition for use according to the invention, in addition to lactic acid, butyric acid, and propionic acid, and/or a salt and/or ester and/or anhydride of these molecules, may also comprise at least one molecule chosen from:
oleic acid,
palmitic acid,
lauric acid,
linoleic acid,
azelaic acid,
famesyl cysteine,
palmitoleic acid,
cholesterol,
thioctic acid,
myristic acid,
orotic acid,
acetic acid,
and combinations thereof,
said molecule(s) potentially being in the form of a salt and/or an ester and/or an anhydride of one or more of these molecules.

Preferentially, the composition for its use comprises the following molecules:
oleic acid,
palmitic acid,
lauric acid,
linoleic acid,
azelaic acid,
famesyl cysteine,
palmitoleic acid,
cholesterol,
thioctic acid,
myristic acid,
orotic acid,
acetic acid,
butyric acid,
lactic acid,
propionic acid
and/or a salt and/or an ester and/or an anhydride of one or more of these molecules.

Advantageously, these molecules each act on different factors, enabling a multi-factorial action on the intestinal microbiota. These molecules help to balance bacterial populations in excess or deficit, notably by reducing intestinal inflammation.

Advantageously, the molecules of the composition for use according to the invention can be conjugated to specific polymers, such as poly-lysine, thereby increasing the efficacy and bioavailability of these molecules.

Thus, according to a particularly preferred embodiment, the composition for use according to the invention comprises at least the following conjugates, each conjugate consisting of a molecule covalently bonded to a poly-lysine:
one or more buyrate-poly-L-lysine conjugates
one or more lactate-poly-L-lysine conjugates
one or more propionate-poly-L-lysine conjugates,

According to one variant, the composition for use according to the invention comprises at least:
A. the following conjugates, each conjugate consisting of a molecule covalently bonded to a polylysine:
one or more oleyl-poly-L-lysine conjugates
one or more palmitic-poly-L-lysine conjugates
one or more lauryl-poly-L-lysine conjugates
one or more azelayl-poly-L-lysine conjugates
one or more palmitoleyl-poly-L-lysine conjugates
one or more thioctyl-poly-L-lysine conjugates
one or more myristyl-poly-L-lysine conjugates
one or more orotyl-poly-L-lysine conjugates
one or more acetate-poly-L-lysine conjugates
one or more buyrate-poly-L-lysine conjugates
one or more lactate-poly-L-lysine conjugates
one or more propionate-poly-L-lysine conjugates
one or more linoleyl-poly-L-lysine conjugates, and
B. famesyl cysteine and cholesterol, and/or an ester of these molecules, encapsulated in micelles, preferentially in micelles formed by one or more of the conjugates listed in list A.

The invention also relates to a method for producing these compositions.

Other features and advantages will emerge from the detailed description of the invention and the following examples.

### BRIEF DESCRIPTION OF THE FIGURES

FIG. 1 is a graph showing a PLS-DA comparing the microbiota of WT and SOD1 mice

FIG. 2 is a graph showing a comparative analysis of the beta diversity of the intestinal microbiota of mice via the MDS—Jaccard method according to treatment.

FIG. 3 is a graph showing a comparative analysis of the beta diversity of the intestinal microbiota of mice via the MDS—Bray Curtis method according to treatment.

FIG. 4 is a graph showing a comparative analysis of the intestinal microbiota of mice according to treatment via PLS-DA.

FIG. 5 is a graph showing an analysis of correlation between WT or SOD1 mice according to bacterial family.

FIG. 6 is a graph showing an analysis of correlation between SOD1 mice treated or untreated with a low dose

### DETAILED DESCRIPTION OF THE INVENTION

### Definitions

For the purposes of the invention, the term “animal” means any animal apart from humans. In particular, it is a mammal.

For the purposes of the invention, the term “amphiphilic conjugate” means a conjugate formed by a hydrophobic molecule X and a hydrophilic polymer Y, the conjugate thus having an amphiphilic character.

For the purposes of the invention, the term “molecule X conjugated to a polymer Y” means a molecule X covalently bonded to a polymer Y, this bond preferentially being an amide, urea or carbamate bond depending on the chemical nature of the molecule X and of the polymer Y.

The term “medical nutrition composition” or “medical nutrition product” or “medical food” or foods for special medical purposes (FSMP) or dietary foods for special medical purposes (DFSMP), within the meaning of the invention, is taken to mean a food with a therapeutic purpose of prevention and/or treatment, used alone or in combination with other therapies. It is a nutritional compound, adapted to a particular clinical situation, likely to constitute the exclusive or partial diet of the patients for whom it is intended.

The term “pathological dysbiosis” within the meaning of the invention means a pathological condition characterized by an imbalance in the distribution of the bacteria of the intestinal microbiota at the level of phyla, classes, orders, families, genera or species, leading to a risk of other diseases of metabolic origin.

For the purposes of this invention, the term “drug” refers to a product that has been granted marketing authorization as a preventive and/or therapeutic agent for a disease.

The term “microbiota” within the meaning of the invention is understood to mean intestinal microbiota.

The term “butyrate-producing bacteria” within the meaning of the invention means bacteria capable of producing butyrate by the anaerobic fermentation of non-digestible food fibers. Butyrate-producing bacteria belong mainly to the phylum Firmicutes.

The term “sulfide-producing bacteria” within the meaning of the invention means bacteria capable of reducing sulfates to sulfides.

The term “alpha diversity” within the meaning of the invention means the measurement of the number of species present in a given sample.

The term “beta diversity” within the meaning of the invention means the measurement of the diversity of species in a given sample.

The term “excess of sulfide-producing bacteria” within the meaning of the invention means a higher-than-normal proportion of sulfide-producing bacteria, preferentially higher than the proportion of sulfide-producing bacteria in an individual without pathological dysbiosis.

The term “deficit in butyrate-producing bacteria” within the meaning of the invention means a lower-than-normal proportion of butyrate-producing bacteria, preferentially lower than the proportion of butyrate-producing bacteria in an individual without pathological dysbiosis.

### Composition for Use Thereof

The invention therefore relates to a composition comprising at least:
lactic acid, and/or a salt and/or ester and/or anhydride of lactic acid,
butyric acid, and/or a salt and/or ester and/or anhydride of butyric acid, and
propionic acid, and/or a salt and/or an ester and/or anhydride of propionic acid, for use in humans or animals for the prevention and/or treatment of pathological dysbiosis of the intestinal microbiota.

Preferentially, the composition is particularly suitable for use in humans for the prevention and/or treatment of pathological dysbiosis of the intestinal microbiota.

Advantageously, the combination of these three molecules guarantees a significant preventive and/or curative effect on pathological dysbiosis of the intestinal microbiota. Lactate, butyrate and propionate are the main short-chain fatty acids produced when fiber is broken down by bacteria in the intestinal microbiota. These three fatty acids are involved in numerous cellular functions and are the main source of energy for the intestinal epithelium, more specifically the epithelial cells of the intestine and colon. Too low a content of short-chain fatty acids can lead to pathological dysbiosis.

In the context of the invention, pathological dysbiosis of the intestinal microbiota is preferentially characterized by an excess of sulfide-producing bacteria and a deficiency of butyrate-producing bacteria.

The presence of an excess of sulfide-producing bacteria is characteristic of pathological dysbiosis. In fact, as the proportion of beneficial microorganisms in the microbiota diminishes, bacterial populations appear that have a harmful effect or produce metabolites that are harmful to the organism. Thus, patients with pathological dysbiosis have high levels of sulfur-reducing bacteria associated with increased production of toxic substances such as hydrogen sulfide, known for its pro-inflammatory activity. The presence of these metabolites causes inflammation and increased intestinal permeability.

The use of a composition according to the invention is thus characterized in the intestinal microbiota by an increase in the proportion of butyrate-producing bacteria and a decrease in the proportion of sulfide-producing bacteria.

Advantageously, reducing the proportion of sulfide-producing bacteria reduces intestinal inflammation by treating pathological dysbiosis. On the other hand, increasing the proportion of butyrate-producing bacteria enables intestinal and colonic epithelial cells to restore membrane permeability. This increase stimulates the microbiota, and in particular the bacterial species that are beneficial to the body. Thus, simultaneous action on butyrate- and sulfide-producing bacteria provides a synergistic effect on pathological dysbiosis of the intestinal microbiota. The composition guarantees along-term effect on the intestinal microbiota by targeting the causes of pathological dysbiosis.

The composition can be used to prevent or treat any disease that has resulted in, or has been caused by, pathological dysbiosis of the intestinal microbiota, preferentially pathological dysbiosis of the intestinal microbiota characterized in the microbiota by an increase in the proportion of sulfide-producing bacteria and a decrease in the proportion of butyrate-producing bacteria, and also preferentially characterized by an increase in intestinal inflammation.

More particularly, the composition comprising at least:
lactic acid, and/or a salt and/or ester and/or anhydride of lactic acid,
butyric acid, and/or a salt and/or ester and/or anhydride of butyric acid, and
propionic acid, and/or a salt and/or an ester and/or anhydride of propionic acid can be used in humans or animals in the prevention and/or treatment of at least one disease associated with a pathological dysbiosis of the intestinal microbiota, of at least one disease associated with a pathological dysbiosis of the intestinal microbiota, in particular wherein the human or animal has an intestinal dysbiosis characterized in the microbiota by an increase in the proportion of sulfide-producing bacteria and a decrease in the proportion of butyrate-producing bacteria, and in particular at least one neurodegenerative disease and/or intestinal disease associated with a pathological dysbiosis of the intestinal microbiota, such as at least one neurodegenerative disease and/or intestinal disease associated with a pathological dysbiosis of the intestinal microbiota wherein the human or animal has an intestinal dysbiosis characterized in the microbiota by an increase in the proportion of sulfide-producing bacteria and a decrease in the proportion of butyrate-producing bacteria.

In the context of the invention, neurodegenerative diseases may be selected from multiple sclerosis, amyotrophic lateral sclerosis (also known as Lou Gehrig's disease), Parkinson's disease and Alzheimer's disease.

Furthermore, the intestinal diseases may be selected from among Crohn's disease, chronic inflammatory bowel diseases, hemorrhagic rectocolitis, irritable bowel syndrome, ulcerative colitis, rheumatoid arthritis, and food intolerance, in particular gluten intolerance.

According to one variant, the composition can be used to treat or prevent autism spectrum disorders or spondyloarthritis, in particular wherein the human or animal gas an intestinal dysbiosis characterized in the microbiota by an increase in the proportion of sulfide-producing bacteria and a decrease in the proportion of butyrate-producing bacteria.

The composition thus treats pathological dysbiosis in particular by relatively increasing the proportion of butyrate-producing bacteria in the intestinal microbiota combined with a decrease in the proportion of sulfide-producing bacteria.

The composition thus acts on the intestinal epithelium by providing short-chain fatty acids such as butyric acid, which are essential for the functioning of epithelial cells. Epithelial cells act on inflammation and treat pathological dysbiosis by promoting the growth of butyrate-producing bacteria while restoring membrane permeability.

The composition can thus be used as a drug, medical nutrition product, or dietary supplement to rebalance the intestinal microbiota by increasing the proportion of sulfide-producing bacteria and decreasing the proportion of butyrate-producing bacteria and/or to prevent and combat diseases arising from or engendered by this pathological imbalance of the intestinal microbiota.

In the context of the invention, sulfide-producing bacteria can be bacteria belonging to the family of Desulfovibrionaceae and/or proteolytic bacteria.

The sulfide-producing bacteria belonging to the Desulfovibrionaceae family may belong to at least one bacterial genus selected from:
Alteridesulfovibrio
Aminidesulfovibrio
Bilophila
Desulfocurvus
Desulfovibrio
Frigididesulfovibrio
Fundidesulfovibrio
Halodesulfovibrio
Humidesulfovibrio
Lawsonia
Maridesulfovibrio
Megalodesulfovibrio
Nitratidesulfovibrio
Oleidesulfovibrio
Paradesulfovibrio
Paucidesulfovibrio
Pseudodesulfovibrio
Solidesulfovibrio.

Preferentially, the sulfide-producing bacteria belong to the family Desulfovibrionaceae, the genus Bilophila, and the species wadworthia.

Thus, the composition according to the invention can be used in humans or animals to reduce the proportion of the bacterial species Bilophila wadworthia in humans or animals with intestinal dysbiosis characterized in the microbiota by an increase in the proportion of sulfide-producing bacteria.

The composition also acts on at least one family of butyrate-producing bacteria selected from:
Lachnospiraeceae
Ruminococcacea

The butyrate-producing bacteria belonging to the Lachnospiraeceae family may belong to at least one bacterial genus selected from:
Lachnospira
Roseburia
Eubacterium
Anaerostipes.

The butyrate-producing bacteria of the Ruminococcacea family may belong to the bacterial genus Faecalibacterium.

In a particularly preferred embodiment, the composition can be used to increase the proportion of bacteria from the family Lachnospiraeceae and decrease the proportion of bacteria from the family Desulfovibriaceae, preferably such that Desulfovibriaceae represent less than 0.01% of the total bacteria present in the intestinal microbiota.

Advantageously, the composition reduces inflammation of the digestive system by restoring sulfide and butyrate concentrations similar to those of a healthy patient.

Preferably, the composition, in addition to lactic acid, butyric acid and propionic acid, and/or a salt and/or ester and/or anhydride of these molecules, also comprises at least one molecule chosen from:
oleic acid,
palmitic acid,
lauric acid,
linoleic acid,
azelaic acid,
famesyl cysteine,
palmitoleic acid,
cholesterol,
thioctic acid,
myristic acid,
orotic acid,
pyruvic acid
acetic acid, and combinations thereof,
said molecules potentially being in the form of a salt and/or an ester and/or an anhydride of one or more of these molecules.

Preferentially the composition comprises at least 2 molecules, in particular at least 3, even more preferentially at least 4 chosen from:
oleic acid,
palmitic acid,
lauric acid,
linoleic acid,
azelaic acid,
famesyl cysteine,
palmitoleic acid,
cholesterol,
thioctic acid,
myristic acid,
orotic acid,
pyruvic acid
acetic acid, and combinations thereof,
said molecules potentially being in the form of a salt and/or an ester and/or an anhydride of one or more of these molecules.

According to a particular embodiment of the invention, the composition, in addition to lactic acid, butyric acid and propionic acid, and/or a salt and/or ester and/or anhydride of these molecules, also comprises at least azelaic acid, cholesterol, myristic acid and lauric acid and/or a salt and/or ester and/or anhydride of these molecules.

For all of the active molecules present in the compositions for use according to the invention, when they are mentioned in the present application, they can be the molecules (e.g. butyric acid) per se and/or a salt (example: butyrate) and/or an ester and/or an anhydride of these molecules (e.g. butyric acid ester) and/or an anhydride.

According to a particularly preferred embodiment of the invention, the composition comprises at least the following molecules:
oleic acid,
palmitic acid,
lauric acid,
linoleic acid,
azelaic acid,
famesyl cysteine,
palmitoleic acid,
cholesterol,
thioctic acid,
myristic acid,
orotic acid,
lactic acid
propionic acid
butyric acid
acetic acid, and combinations thereof,
said molecule(s) potentially being in the form of a salt and/or an ester and/or an anhydride of one or more of these molecules.

Such a composition, surprisingly and unexpectedly, acts on the intestinal microbiota and in particular is able to increase the proportion of butyrate-producing bacteria and decrease the proportion of sulfide-producing bacteria.

The useful composition according to the invention comprising at least all of the following molecules:
oleic acid,
palmitic acid,
lauric acid,
linoleic acid,
azelaic acid,
famesyl cysteine,
palmitoleic acid,
cholesterol,
thioctic acid,
myristic acid,
orotic acid,
lactic acid
propionic acid
butyric acid
acetic acid, and combinations thereof,
said molecule(s) potentially being in the form of a salt and/or an ester and/or an anhydride of one or more of these molecules, is hereafter referred to as composition C1.

The useful composition according to the invention, and in particular composition C1, can optionally also comprise pyruvic acid and/or a salt and/or an ester and/or an anhydride.

### Preferentially:

the amount of butyric acid in the composition, and more particularly in composition C1, is greater than that of each of the other molecules taken individually, and/or
the amount of thioctic acid in the composition, and more particularly in composition C1, is greater than that of each of the other molecules taken individually, except butyric acid, and/or
the amount of lauric acid in the composition, and more particularly in composition C1 is greater than that of each of the following molecules taken individually: palmitic acid, linoleic acid, azelaic acid, famesyl cysteine, palmitoleic acid, cholesterol, myristic acid, and orotic acid,
the amount of oleic acid on the one hand, and of lactic acid on the other hand, in the composition, and more particularly in composition C1 is greater than that of each of the following molecules taken individually: palmitic acid, lauric acid, linoleic acid, azelaic acid, famesyl cysteine, palmitoleic acid, cholesterol, myristic acid, orotic acid, and acetic acid.

Preferentially, each of the molecules in composition C1 represents between 0.5 E-05 M and 10 E-05 M.

According to a particular embodiment of the invention, the useful composition according to the invention, and in particular composition C1, can be used by administering a dose of between 0.5 and 10 mg/mL, in particular between 2 and 8 mg/mL, preferentially 7 mg/mL to treat pathological dysbiosis of the intestinal microbiota.

According to one variant, the useful composition according to the invention, and in particular composition C1, is administered with a first “attack” dose of between 7 and 10 mg/mL, preferentially between 9 and 10 mg/mL, followed by a “maintenance” dose of preferentially between 0.5 and 5 mg/mL, in particular between 2 and 3 mg/mL.

Preferably, the useful composition according to the invention, and in particular C1, may comprise at least one polymer selected from poly-lysine, poly-ethylene glycol, poly-omithine, poly-arginine and poly-histidine.

More particularly, in order to improve the solubility, efficacy and bioavailability of the molecules of the useful composition according to the invention, at least one of the molecules of the composition selected from among butyric acid, lactic acid, propionic acid, salts of these acids, esters of these molecules, and anhydrides of these acides, is covalently conjugated to at least one molecule of a polymer selected from among polylysine, polyethylene glycol, polyomithine, polyarginine and polyhistidine.

When the composition is composition C1 or a composition comprising molecules from the below list, preferentially at least one of the molecules of the composition selected from among oleic acid, palmitic acid, lauric acid, linoleic acid, azelaic acid, palmitoleic acid, thioctic acid, myristic acid, orotic acid, acetic acid, butyric acid, lactic acid, propionic acid, salts of these acids, esters of these acids and anhydrides of these acids, is covalently conjugated to at least one molecule of a polymer selected from among polylysine, polyethylene glycol, polyomithine, polyarginine and polyhistidine.

Specifically, the molecules selected from among oleic acid, palmitic acid, lauric acid, linoleic acid, azelaic acid, palmitoleic acid, thioctic acid, myristic acid, orotic acid, acetic acid, butyric acid, lactic acid, propionic acid, salts of these acids, esters of these acids and anhydrides of these acids can all be covalently conjugated to a polymer selected from among polylysine, polyethylene glycol, polyomithine, polyarginine and polyhistidine, in particular via an amide, urea or carbamate bond.

According to one particularly suitable variant, all of the molecules selected from among oleic acid, palmitic acid, lauric acid, linoleic acid, azelaic acid, palmitoleic acid, thioctic acid, myristic acid, orotic acid, acetic acid, butyric acid, lactic acid, propionic acid, salts of these acids and esters of these acids, when present in composition according to invention, are covalently conjugated to at least one molecule of a polymer selected from among polylysine, polyethylene glycol, polyomithine, polyarginine and polyhistidine.

The useful composition according to the invention may comprise micelles. When the composition according to the invention, and in particular composition C1, comprises famesyl cysteine and/or cholesterol and/or one of their esters and/or salts and/or anhydrides, the useful composition according to the invention preferably comprises micelles wherein are encapsulated at east famesyl cysteine and/or cholesterol and/or one of their esters and/or salts and/or anhydrides.

According to one embodiment, the useful composition according to the invention preferentially comprises:
conjugates of oleic acid, palmitic acid, lauric acid, linoleic acid, azelaic acid, palmitoleic acid, thioctic acid, myristic acid, orotic acid, acetic acid, butyric acid, lactic acid, propionic acid, salts of these acids, esters of these acids and anhydrides of these acids with polylysine, polyethylene glycol, polyomithine, polyarginine or polyhistidine, and
micelles formed by at least one of these conjugates, said micelles encapsulating famesyl cysteine and cholesterol.

Preferentially, at least one of the micelles is formed by amphiphilic conjugates each consisting of at least one hydrophobic molecule covalently conjugated to a molecule of a polymer selected from among polylysine, polyethylene glycol, polyomithine, polyarginine and polyhistidine.

Thus, according to one particular embodiment of the invention, at least one micelle is formed by amphiphilic conjugates each consisting of at least one molecule selected from among oleic acid, palmitic acid, lauric acid, linoleic acid, palmitoleic acid, myristic acid, salts, esters and anhydrides of these fatty acids, covalently conjugated to a molecule of a polymer selected from among polylysine, polyethylene glycol, polyomithine, polyarginine and polyhistidine.

In particular, this configuration makes it possible:
to increase the half-life of the active molecules of the composition in the body,
to target tissues or cells on which the molecules of the composition are to act,
to increase the stability and bioavailability of the active ingredient.

The efficacy of the composition is therefore enhanced and it is possible to administer lower doses and reduce the acute or chronic toxicity of the active molecules contained in the composition.

Preferentially, the one or more polymers conjugated to the molecules are selected from among poly-L-lysine, polyethylene glycol, poly-L-omithine, poly-L-arginine and poly-L-histidine.

The polylysine used in the compositions according to the invention is preferentially a poly-L-lysine. The polylysine is preferentially linear. In particular, the polylysine used can be an epsilon poly-L-lysine, preferentially a poly-L-lysine with a molecular weight of between 12,000 and 20,000 Da. The polylysine used in the compositions according to the invention can be a polylysine that has bromide, chloride or TFA, trifluoroacetic acid, counterion.

Thus, according to one variant, composition C1 comprises at least:
A: the following conjugates, each conjugate consisting of a molecule covalently bonded to a polylysine:
one or more oleyl-poly-L-lysine conjugates
one or more palmitic-poly-L-lysine conjugates
one or more lauryl-poly-L-lysine conjugates
one or more azelayl-poly-L-lysine conjugates
one or more palmitoleyl-poly-L-lysine conjugates
one or more thioctyl-poly-L-lysine conjugates
one or more myristyl-poly-L-lysine conjugates
one or more orotyl-poly-L-lysine conjugates
one or more acetate-poly-L-lysine conjugates
one or more buyrate-poly-L-lysine conjugates
one or more lactate-poly-L-lysine conjugates
one or more propionate-poly-L-lysine conjugates
one or more linoleyl-poly-L-lysine conjugates,
B: famesyl cysteine and cholesterol, and/or an ester of these molecules, encapsulated in micelles, preferentially in micelles formed by one or more of the conjugates listed in list A.

In this composition, the poly-L-lysine can be replaced with another polylysine or with polyethylene glycol, a poly-L-omithine, a poly-L-arginine or a poly-L-histidine.

The composition for use according to the invention can be in solid form or in liquid form. When in liquid form, the composition comprises at least water and the constituents mentioned above. The solid form is preferentially obtained from the liquid form, preferentially by freeze-drying. When the composition is in liquid form and comprises micelles and it is freeze-dried into solid form, the micelles reform when the composition in solid form is placed back into an aqueous solution.

The composition for use according to the invention also preferentially comprises at least one pharmaceutically acceptable excipient. The excipient can be selected in particular to meet the pH and osmolarity requirements of solutions for injection into humans or animals. For example, they can be acids or bases for adjusting pH or NaCl for adjusting osmolarity.

The composition for use according to the invention, in particular composition C1, is intended to be administered to a human, or to an animal, and is consequently in a form suitable for such administration. When it is in liquid form, it is preferentially suitable for subcutaneous or intravenous administration, in particular intravenous infusion, and it is packaged in suitable containers known to a person skilled in the art for packaging this type of product. The composition according to the invention can also be administered in liquid form via a pump, like an insulin pump.

When it is in solid form, it is preferentially suitable for administration:
transcutaneously and it is preferentially formulated and packaged in the form of a patch, or
nasally in the form of a powder, or
sublingually in the form of a powder or tablet, or
transmucosally, and it is preferentially formulated and packaged in the form of a mucoadhesive tablet.

### Manufacturing Method

The useful composition according to the invention can be produced using any suitable method.

If the molecules that constitute the composition are used as they are in a solvent, they can all be mixed together in said solvent.

If the molecules in the composition are conjugated to polymers for some and in micelles for others, the production method can comprise the following steps:
a. creating an amphiphilic premix: mixing, in an aqueous solution:
one or more buyrate-poly-L-lysine conjugates
one or more lactate-poly-L-lysine conjugates
one or more propionate-poly-L-lysine conjugates
b. optionally, adding at least famesyl cysteine and cholesterol to the amphiphilic premix and stirring so as to form micelles formed by one or more of the conjugates of the amphiphilic premix encapsulating the famesyl cysteine and cholesterol.

Preferably, the manufacturing method may comprise the following steps:
a. creating an amphiphilic premix: mixing, in an aqueous solution:
one or more buyrate-poly-L-lysine conjugates
one or more lactate-poly-L-lysine conjugates
one or more propionate-poly-L-lysine conjugates, and
at least one or more conjugates selected from:
one or more oleyl-poly-L-lysine conjugates
one or more palmitic-poly-L-lysine conjugates
one or more lauryl-poly-L-lysine conjugates
one or more azelayl-poly-L-lysine conjugates
one or more palmitoleyl-poly-L-lysine conjugates
one or more thioctyl-poly-L-lysine conjugates
one or more myristyl-poly-L-lysine conjugates
one or more orotyl-poly-L-lysine conjugates
one or more acetate-poly-L-lysine conjugates
one or more linoleyl-poly-L-lysine conjugates, and
b. adding at least famesyl cysteine and cholesterol to the amphiphilic premix and stirring so as to form micelles formed by one or more of the conjugates of the amphiphilic premix encapsulating the famesyl cysteine and cholesterol.

One variant of the invention consists in taking advantage of the amphiphilic nature of the majority of the individual components to create an amphiphilic premix which allows the controlled solubilization of the hydrophobic species. The solubilization process according to one preferred embodiment consists in the controlled addition of the hydrophobic molecules to the amphiphilic premixes and allowing the time needed for their dissolution with stirring.

The poly-L-lysine can be replaced with another polylysine or with polyethylene glycol, a poly-L-omithine, a poly-L-arginine or a poly-L-histidine.

Preferentially, the stirring is carried out for at least 60 minutes, even more preferentially between 5 and 20 minutes, and preferably at a stirring speed of 900 revolutions per minute or less, in particular at a stirring speed of between 50 and 800 revolutions per minute.

According to one preferred embodiment, the production method according to the invention also comprises a step c. of separating the soluble and insoluble phases, in order to recover the soluble phase. In this case, the insoluble phase is removed and the soluble phase constitutes the composition according to the invention. Specifically, a physical separation process (filtration, ultrafiltration) is preferentially carried out to ensure the isolation of the soluble fraction containing both the amphiphilic premix and the solubilized molecules.

The composition in liquid form can then be freeze-dried or dehydrated to be in solid form, preferentially by means of slow freeze-drying, for example between 12 and 36 hours.

Moreover, if active molecule-polymer conjugates of the composition are not incorporated into the premix in step a, they can be added to the mixture after step b., that is, after the formation of micelles and the encapsulation of famesyl cysteine and cholesterol.

The active molecule-polymer conjugates can be produced by any means known to a person skilled in the art for conjugating a molecule covalently to a polymer depending on their chemical nature. Thus, lauric acid, myristic acid, palmitic acid, oleic acid, linoleic acid, orotic acid, azelaic acid, thioctic acid, acetic acid, palmitoleic acid, butyric acid, lactic acid, propionic acid and oleic acid are conjugated to the polymer (preferentially poly-L-lysine) via an amide bond.

Exemplary embodiments of amide, urea and carbamate bonds are described, for example, in:
Ryser H J, Shen W C. Conjugation of methotrexate to poly (L-lysine) as a potential way to overcome drug resistance. Cancer. 1980 Mar 15; 45(5 Suppl): 1207-11 (amide bonds),
Zhuxian Z, Jianbin T, Qihang S, William J. A multifunctional PEG-PLL drug conjugate forming redox-responsive nanoparticles for intracellular drug delivery, Issue 38, 2015. Journal of Materials Chemistry B (amide bonds),
Scheper V, Wolf M, Scholl M, Kadlecova Z, Perrier T, Klok H A, Saulnier P, Lenarz T, Stover T. Potential novel drug carriers for inner ear treatment: hyperbranched poly-lysine and lipid nanocapsules. Nanomedicine (Lond). 2009 Aug; 4(6):623-35 (urea bonds),
Stéphanie Gac-Breton, Jean Coudane, Mahfoud Boustta & Michel Vert (2004) Norfloxacin-Poly(I-Lysine Citramide Imide) Conjugates and Structure-dependence of the Drug Release, Journal of Drug Targeting, 12:5, 297-307, (carbamate bonds),
Elmore, W. M. (2013). Nanoparticles Stabilized with MPEG-Polylysine Carbamate: Synthesis and Characterization, (carbamate bonds),
Ning-Ping Huang, Janos Vörös, Susan M. De Paul, Marcus Textor, and Nicholas D. Spencer. Biotin-Derivatized Poly(l-lysine)-g-poly(ethylene glycol): A Novel Polymeric Interfacefor Bioaffinity Sensing. Langmuir 2002 18 (1), 220-230 (carbamate bonds).

### EXAMPLES

Example of a composition suitable for use according to the invention:

### Example 1

A suitable exemplary composition according to the invention is presented below.

TABLE 1

| PLL conjugate | Concentration |
|---|---|
| (PLL = poly-L-lysine) | (mg/mL) |
| Buyrate-PLL | 1.16 |
| Lactate-PLL | 0.73 |
| Propionate-PLL | 0.72 |

Each conjugate was weighed in a sterile 50 ml centrifuge tube, and was dissolved in 30 ml of water, using a vortex mixer. Each conjugate was stirred for a period of between 10 and 20 minutes depending on the solubility of the conjugate.

### Example 2

A suitable exemplary composition according to the invention is presented below.

TABLE 2

| PLL conjugate | Concentration |
|---|---|
| (PLL = poly-L-lysine) | (mg/mL) |
| Buyrate-PLL | 0.89 |
| Lactate-PLL | 0.56 |
| Propionate-PLL | 0.55 |

Each conjugate was weighed in a sterile 50 ml centrifuge tube, and was dissolved in 30 ml of water, using a vortex mixer. Each conjugate was stirred for a period of between 10 and 20 minutes depending on the solubility of the conjugate.

### Example 3

A suitable exemplary composition according to the invention is presented below.

TABLE 3

| PLL conjugate | Concentration |
|---|---|
| (PLL = poly-L-lysine) | (mg/mL) |
| Buyrate-PLL | 1.59 |
| Lactate-PLL | 1 |
| Propionate-PLL | 0.98 |

Each conjugate was weighed in a sterile 50 ml centrifuge tube, and was dissolved in 30 ml of water, using a vortex mixer. Each conjugate was stirred for a period of between 10 and 20 minutes depending on the solubility of the conjugate.

### Example 4

An exemplary composition suitable for testing on mice is presented below.

TABLE 4

| PLL conjugate | Concentration |
|---|---|
| (PLL = poly-L-lysine) | (mg/mL) |
| Buyrate-PLL | 0.089 |
| Lactate-PLL | 0.056 |
| Propionate-PLL | 0.055 |

Each conjugate was weighed in a sterile 50 ml centrifuge tube, and was dissolved in 30 ml of water, using a vortex mixer. Each conjugate was stirred for a period of between 10 and 20 minutes depending on the solubility of the conjugate.

### Example 5

A suitable exemplary composition according to the invention is presented below.

TABLE 5

| PLL conjugate | Concentration |
|---|---|
| (PLL = poly-L-lysine) | (mg/mL) |
| Buyrate-PLL | 1.16 |
| Lactate-PLL | 0.73 |
| Propionate-PLL | 0.72 |
| Thioctyl-PLL | 0.92 |

Each conjugate was weighed in a sterile 50 ml centrifuge tube, and was dissolved in 30 ml of water, using a vortex mixer. Each conjugate was stirred for a period of between 10 and 20 minutes depending on the solubility of the conjugate.

### Example 6: Exemplary Composition C1

A useful exemplary composition C1 according to the invention is presented below.

The following amounts of conjugates were weighed in order to prepare a stock of 300 ml of composition C1. All of the conjugates were weighed using an analytical balance under a laminar flow hood.

TABLE 6

| PLL conjugate | Concentration 1X |
|---|---|
| (PLL = poly-L-lysine) | (mg/mL) |
| Oleyl-PLL | 0.82 |
| Palmitic-PLL | 0.32 |
| Lauryl-PLL | 0.78 |
| LinoleyI-PLL | 0.32 |
| Azelayl-PLL | 0.31 |
| Palmitoleyl-PLL | 0.64 |
| Thioctyl-PLL | 0.92 |
| Myristyl-PLL | 0.32 |
| Orotyl-PLL | 0.3 |
| Acetate-PLL | 0.72 |
| Buyrate-PLL | 1.16 |
| Lactate-PLL | 0.73 |
| Propionate-PLL | 0.72 |

Each conjugate was weighed in a sterile 50 ml centrifuge tube, and was dissolved in 30 ml of water, using a vortex mixer. Each conjugate was stirred for a period of between 10 and 20 minutes depending on the solubility of the conjugate.

In parallel, cholesterol and famesyl cysteine molecules were dissolved separately at the approximate concentration of 50 mg/mL in absolute ethanol and filtered over a 0.22 μm filter. The solvent was evaporated using a rotary evaporator. The required amounts of dry solids were weighed under a laminar flow hood, as presented in table 3.

TABLE 7

| | Concentration 1X |
|---|---|
| Molecule | (mg/mL) |
| Cholesterol-PLL | 0.33 |
| farnesyl cysteine-PLL | 0.32 |

In a sterile 500 ml flask equipped with a magnetic bar, 23.8 ml of each conjugated solution prepared previously was added while being magnetically stirred (700 revolutions per minute). The corresponding amounts of (previously freeze-dried) salts at 300 of PBS were added to the solution of conjugates while being magnetically stirred (700 revolutions per minute). Cholesterol and famesyl cysteine were added and the mixture was stirred for 25 hours (700 revolutions per minute).

30 ml of formulation C1 10× was taken under a laminar flow hood and added to a new sterile 500 ml flask.

270 ml of sterile PBS was added to obtain formulation C1 1×. The formulation was stirred for 30 minutes.

### Example 7: Exemplary Composition C1

An exemplary composition C1 suitable for testing on mice is presented below.

The following amounts of conjugates were weighed in order to prepare a stock of 300 ml of composition C1. All of the conjugates were weighed using an analytical balance under a laminar flow hood.

TABLE 8

| PLL conjugate | Concentration 1X | Concentration 10X |
|---|---|---|
| (PLL = poly-L-lysine) | (mg/mL) | (mg/mL) |
| Oleyl-PLL | 0.063 | 0.63 |
| Palmitic-PLL | 0.024 | 0.24 |
| Lauryl-PLL | 0.059 | 0.59 |
| Linoleyl-PLL | 0.025 | 0.25 |
| Azelayl-PLL | 0.024 | 0.24 |
| Palmitoleyl-PLL | 0.049 | 0.49 |
| Thioctyl-PLL | 0.07 | 0.7 |
| Myristyl-PLL | 0.024 | 0.24 |
| Orotyl-PLL | 0.023 | 0.23 |
| Acetate-PLL | 0.055 | 0.55 |
| Buyrate-PLL | 0.089 | 0.89 |
| Lactate-PLL | 0.056 | 0.56 |
| Propionate-PLL | 0.055 | 0.55 |

Each conjugate was weighed in a sterile 50 ml centrifuge tube, and was dissolved in 30 ml of water, using a vortex mixer. Each conjugate was stirred for a period of between 10 and 20 minutes depending on the solubility of the conjugate.

In parallel, cholesterol and famesyl cysteine molecules were dissolved separately at the approximate concentration of 50 mg/ml in absolute ethanol and filtered over a 0.22 μm filter. The solvent was evaporated using a rotary evaporator. The required amounts of dry solids were weighed under a laminar flow hood.

TABLE 9

| | Concentration 1X | Concentration 10X |
|---|---|---|
| Molecule | (mg/mL) | (mg/mL) |
| Cholesterol-PLL | 0.004 | 0.04 |
| farnesyl cysteine-PLL | 0.004 | 0.04 |

In a sterile 500 ml flask equipped with a magnetic bar, 23.8 ml of each conjugated solution prepared previously was added while being magnetically stirred (700 revolutions per minute). The corresponding amounts of (previously freeze-dried) salts at 300 of PBS were added to the solution of conjugates while being magnetically stirred (700 revolutions per minute).

Cholesterol and famesyl cysteine were added and the mixture was stirred for 25 hours (700 revolutions per minute).

30 ml of formulation C1 10× was taken under a laminar flow hood and added to a new sterile 500 ml flask.

270 ml of sterile PBS was added to obtain formulation C1 1×. The formulation was stirred for 30 minutes.

The flasks were prepared according to the following plan using a sterile calibrated 5 ml pipette:
C1 1×=83 flasks, 2 ml each.
C1 10×=83 flasks, 2 ml each.

The 166 flasks were placed in a steel freeze-drying unit and a freeze-drying cycle of one day was initiated.

### Example 8: Molecular Analysis of Mouse Fecal Microbiota (Wild-Type—WT, SOD1, SOD1+Composition According to the Invention)

The SOD1 mouse is an animal model for axon degeneration consisting of transgenic mice expressing the mutated form of the human gene for superoxide dismutase. It is the in-vivo reference model for studying amyotrophic lateral sclerosis.

In this test, compositions C1 (described in table 8 and 9) and C2 (described in table 10) were injected one after the other with a 1-hour interval, starting with composition C1.

TABLE 10

| PLL conjugate | Concentration 1X | Concentration 10X |
|---|---|---|
| (PLL = poly-L-lysine) | (mg/mL) | (mg/mL) |
| Alpha-tocopherol-PLL | 0.070 | 0.70 |
| Ascorbyl PLL | 0.063 | 0.63 |
| Biotinyl-PLL | 0.068 | 0.68 |
| GABA-PLL | 0.126 | 1.26 |
| Glutathione-PLL | 0.074 | 0.74 |
| Pantothenyl-PLL | 0.068 | 0.68 |
| Retynoil-PLL | 0.202 | 2.02 |
| Spermine-PLL | 0.066 | 0.66 |
| Orotyl-PLL | 0.110 | 1.10 |
| PLL/Cysteine (copolymer) | 0.06 | 0.6 |
| PLL/Methionine (copolymer) | 0.101 | 1.01 |

The feces of five male and female mice from each group (WT, SOD1, SOD1+C1 low dose and SOD1+C1 high dose), collected from the cages, were analyzed at T0 (=T6), T3 weeks (T9) and 10 weeks (T16), that is, a total of 55 samples.

In a first step, the total genomic DNA was extracted from each fecal sample using Godon's method (Godon et al, Appl Envion. Microbiol, 1997). The quality of the extracted DNA was evaluated after migration of the samples through agarose gel. DNA concentration was then determined using NanoDrop technology.

The DNA samples obtained were then sequenced. The approach used was high-throughput sequencing of the gene coding for 16S ribosomal RNA (V3-V4 region) using Illumina technology (read lengths: 150 bases, read depth: 1 million reads).

The taxonomic affiliation of each bacterial sequence (or OTU) obtained was carried out using the silva_nr99_vl38.1 (https://www.arb-silva.de/) and GTDB v202 (https://gtdb.ecogenomic.org/) databases.

The rANOMALY pipeline (Theil S and Rifa E. rANOMALY: AmplicoN wOrkflow for Microbial community AnaLYsis [version 1; peer review: 2 approved]m F1000Research 2021, 10:7 https://doi.org/10.12688/f1000research.27268.1), based on the DADA2 program, was used to process all of the sequences, whether to estimate the abundance of bacterial phyla/families or to carry out statistical analyses (composition, diversity analyses, differential abundance analyses).

The comparison of bacterial diversity and richness between the microbiota obtained from treated and untreated mice was carried out by calculating the Shannon index. The structural differences of the populations (beta diversity) were revealed using two multivariate analysis methods (ANOVA and non-metric multidimensional scaling=MDS method) and a PLS-DA (partial least squares discriminant analysis) from the mixOmics packages.

The OTU differential abundance analyses were carried out using the normalization and estimation tools from the DESeq2 package [Love, M. I., Huber, W., Anders, S. Moderated estimation of fold change and dispersion for RNA-seq data with DESeq2 Genome Biology 15(12):550; 2014],

### Microbiota Correlation—Clinical Parameters

The potential correlation between each of the measured clinical and physiological parameters and the molecular composition of the microbiota was studied for each of the female mice for which the microbiota was analyzed (five mice per group).

Several analysis methods were tested, based on rank correlation (Spearman's rank correlation).

The method adopted is canonical correlation analysis (rCCA) provided by the mixOmics package (Rohart F, Gautier B, Singh A, and Le Cao K-A, 2017: mixOmics: An R package for 'omics feature selection and multiple data integration. PLoS computational biology 13(11):e1005752). This method makes it possible to perform cross-validation on the results of analyses of the correlations between the abundance of OTUs and the various clinical parameters measured. The results obtained are presented in the form of heat map showing significant, positive or negative, correlations between the abundance of OTUs and the clinical parameters.

### Results

Comparison of the Composition of the Fecal Microbiota of “SOD1” KO Mice with “Wild-Type” (WT) Mice

The analysis of the relative abundance of phyla, orders, families or genera of bacteria present in the SOD1 mice relative to the WT mice shows that the composition of the SOD1 microbiota differs from that of the WTs:
the SOD1s have fewer Firmicutes and more Bacteroidetes than the WTs Among Bacteroidetes, the SOD1 mice host more Bacteroidales, in particular Muribaculaceae and more Porphyromonadaceae, including Muribaculum, than the WTs
Among Firmicutes, the relative abundance of Lachnospiraceae (Shaedlerella and Acetifractor) and Erysipelotrichales is decreased whereas Lactobacillales such as Limolactobacillus and Oscillospiraceae, as well as Eggerthellaceae, show increased abundance relative to the WT mice.

Analysis of the diversity (alpha diversity) and richness (beta diversity) of the fecal microbiota of the SOD1 mice in comparison with that of the WT mouse made it possible to determine that there is:
no significant difference in bacterial diversity (index: Chao1, Observed, Shannon and Inv Simpson indexes) between SOD1 and WT (alpha diversity).
A significant difference (Jaccard: p=0.01 and Bray-Curtis: p=0.01) in richness of bacterial species (beta diversity). No time effect (T0, T6 and T16) was demonstrated.

The Jaccard and Bray-Curtis indexes or distances are two well-known metrics for measuring similarity, dissimilarity and diversity between multiple samples.

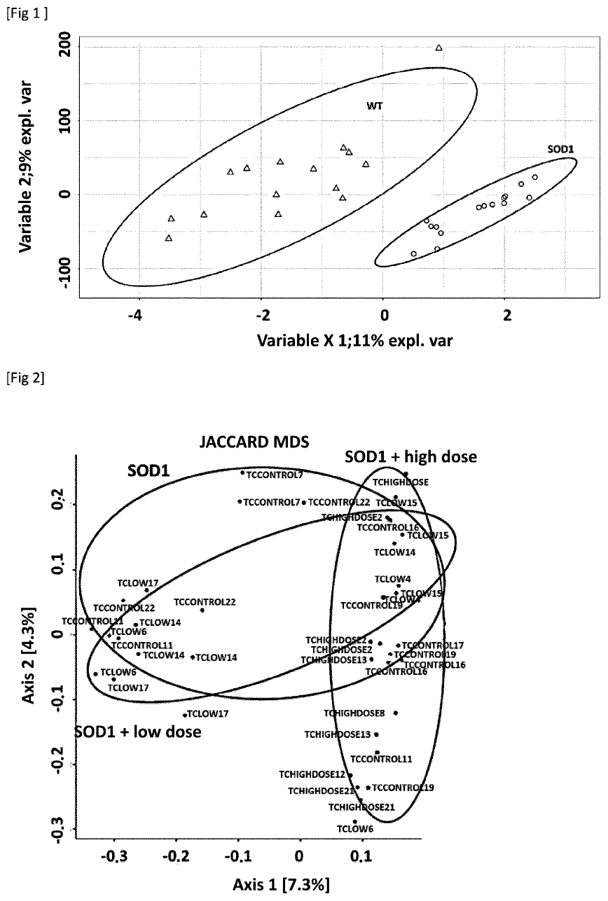

Lastly, the PLS-DA analysis showed that the fecal microbiota of the WT and SOD1 mice differ (FIG. 1).

In conclusion, SOD1 mice had disrupted intestinal microbiota compared with WT mice, characterized by less bacterial richness, a greater proportion of gram-negative bacteria (Muribaculaceae and Muribaculum) as well as gram-positive bacteria from Lactobacillales and Eggerthellaceae, but a lower proportion of Lachnospiraceae and Erysipelotrichales.

Result: Effect of the Administration of Composition C1 (Combined with C2) at High or Low Dose in SOD1 Mice
A—Administration of a 10× high dose of C1 (combined with C2)

Compositions C1 and C2 are described in Tables 7 to 10.

Administration of the composition C1 (combined with C2) at a high dose for 3 weeks leads to:
An increase in the relative abundance of:
Bacteroidales, in particular Muribaculaceae
Oscillospiraceae
COE1
A decrease in the abundance of:
Lactobacillaceae (Lactobacillus genus)
Lachnospiraceae

Administration of a high dose of the composition C1 for 3 weeks appears to accentuate the disruptions observed in SOD1 mice.
B—Administration of a 1× low dose of C1 (combined with C2)

Administration of a low dose of C1 (combined with C2) to SOD1 mice also caused changes in the relative abundance of certain microbial groups. Thus, the following were observed:
An increase in the relative abundance of:
Lachnospiraceae
Oscillospiraceae
GAC-485
A decrease in the relative abundance of:
Lactobacillales

The low dose seems to partly rebalance the intestinal microbiota of the SOD1 mice so as to approximate the composition of the microbiota of the wild-type (WT) mice, in particular by bringing about an increase in Lachnospiraceae and a decrease in Lactobacillales. This effect seems progressive since it is more pronounced after 10 weeks of treatment than after three weeks.

### Result: Comparison of Bacterial Diversity and Richness in Treated and Untreated SOD1 Mice.

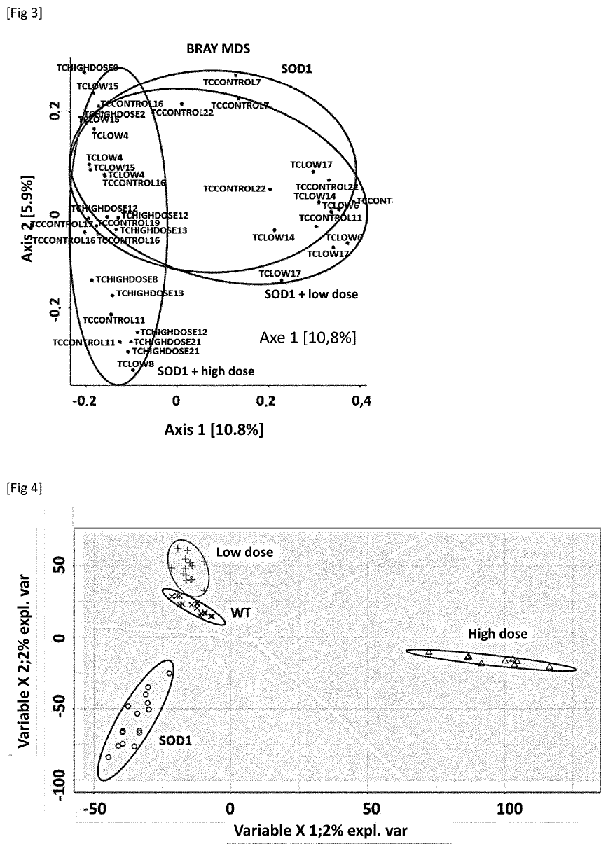

Analysis of the alpha diversity of the fecal microbiota did not demonstrate a significant effect from the administration of C1 (combined with C2), at low and high doses, on bacterial diversity in SOD1 mice. A significant difference (Shannon index, p=0.02) was only observed between the fecal microbiota of mice treated with the low dose and those treated with the high dose of C1 (combined with C2) (FIGS. 2 and 3).

Analysis of beta diversity made it possible to show significant differences between untreated SOD1 mice and treated SOD1 mice:
Controls versus C1 (combined with C2) high dose (p=0.02 Jaccard and p=0.01 Bray-Curtis)
C1 (combined with C2) low dose versus C1 (combined with C2) high dose (p=0.02 Jaccard and p=0.01 Bray-Curtis)
No significant difference between untreated control and C1 (combined with C2) low dose.
   Results: Comparison Between Wild-Type Mice and SOD1 Mice Treated or Untreated with Different Doses of C1 (Combined with C2)

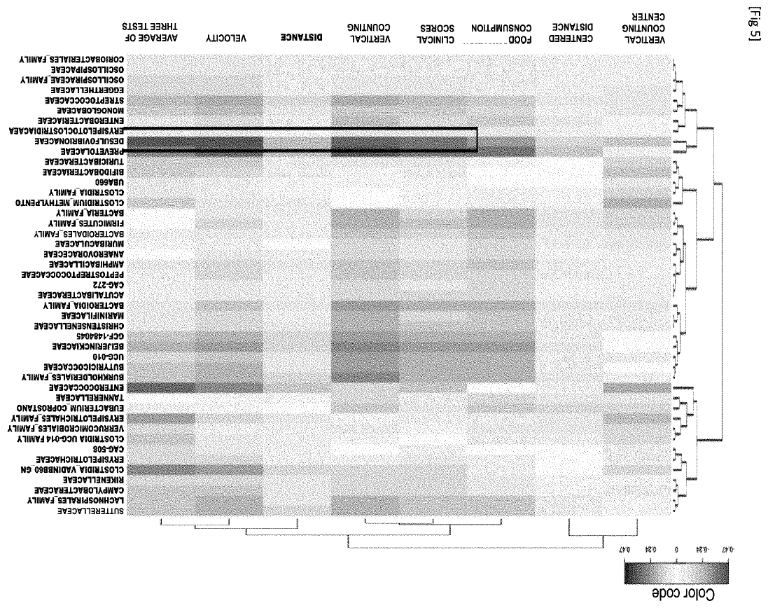

PLS-DA of the composition of all of the microbiota studied made it possible to show that these microbiota could be separated into 4 distinct groups, with the microbiota of the mice treated with the low dose of C1 (combined with C2) coming close to-being in the same cluster as—the microbiota of the wild-type mice (FIG. 4).

### Conclusion

The study made it possible to show that the composition of the microbiota of the SOD1 mice differs from that of the wild-type mice, with these alterations affecting microbiota richness more than microbiota diversity. The microbiota of the SOD1 mice appears to be disrupted since the relative abundance of certain major bacterial groups is altered. It is characterized, in particular, by a higher abundance of Muribaculaceae and Lactobacillales (Oscillospiraceae) at the expense of Lachnospiraceae and Erysipelotrichales. The PLS-DA additionally demonstrated that the microbiota of wild-type and SOD1 mice were distinct.

The administration of a high dose of C1 (combined with C2) to the SOD1 mice for 3 weeks appears to amplify the differences observed between the wild-type mice and SOD1 mice (increase in Muribaculaceae and Oscillospiraceae and decrease in Lachnospiraceae). The composition of the microbiota of the SOD1 mice treated with the high dose of C1 (combined with C2) is very clearly different from those of the SOD1 controls as well as those of the wild-type mice, this group of mice treated with the high dose being substantially separated from the others (most disrupted microbiota).

The administration of a low dose of C1 (combined with C2) to the SOD1 mice for 3 to 10 weeks appears to partly rebalance the disrupted microbiota observed in the SOD1 control mice. Specifically, an increase in Lachnospiraceae and a decrease in Lactobacillales microbial groups is observed in the SOD1 mice relative to the wild-type mice. The composition of the microbiota of the SOD1 mice treated with a low dose of C1 (combined with C2) thus comes close to that of the wild-type mice, the microbiota of these two groups being grouped in the same cluster.

These preliminary results are of interest with regard to the administration of the lowest dose in the SOD1 model mouse. Specifically, at this dose, C1 (combined with C2) seems to have a beneficial effect on the disrupted microbiota of the SOD1 mice.

### Example 9: Study on Correlation Between Symptoms and Molecular Composition of the Microbiota in SOD1 Mice after Administration of a Low Dose of C1 (Combined with C2)

The objective of the study was to analyze potential correlations between the molecular composition of the intestinal microbiota and the clinical parameters measured in the SOD1 mice versus the wild-type mice and in the SOD1 mice treated or untreated with a low dose of C1 (combined with C2) for 10 weeks.

This study was carried out in female mice in which the molecular analysis of the fecal microbiota thereof was carried out beforehand (five females from each group [WT, SOD1, SOD1-low dose), collected from the cages, at T0 (=T6), T3 weeks (T9) and 10 weeks (T16)].

Tests were carried out at least 1 hour after the first daily dose of the test or vehicle compound.

A session for one day comprised a 5 min practice run at 4 rpm on a rotary apparatus (AccuScan Instruments, Columbus, USA). One hour later, the animals were tested for three consecutive acceleration trials of 6 min with the speed going from 0 to 40 rpm over 360 s, with an inter-trial interval of at least 30 min. The latency to fall from the rod was recorded

### Study on Correlation in SOD1 and Wild-Type Mice.

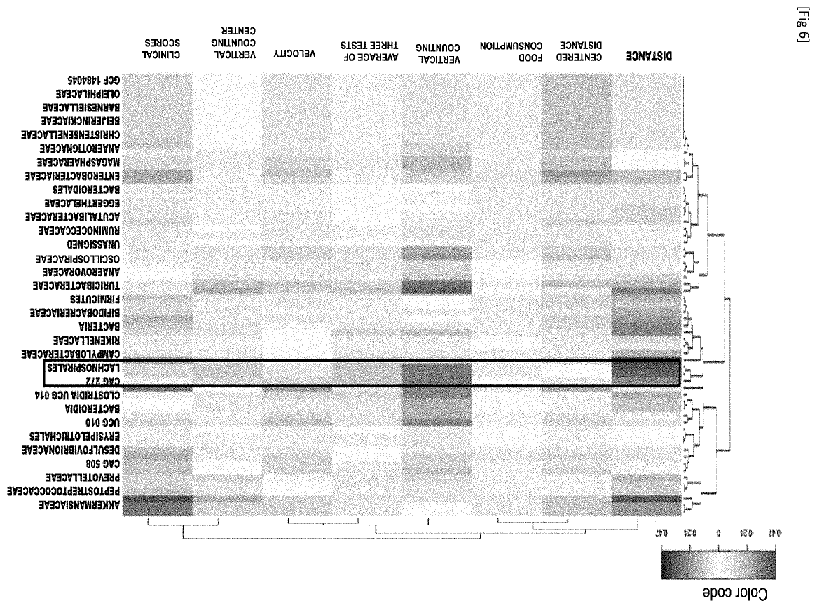

The RCCA (regularized canonical correlation analysis) mainly made it possible to show the presence of a significant negative correlation between clinical score, distance traveled and recovery time after Rotarod and the Desulfovibrionaceae family and a smaller negative correlation with the Prevotellaceae family in WT and SOD1 mice. Thus, the higher the abundance of these bacterial families, the lower the clinical score, and likewise for distance traveled and recovery time (FIG. 5).

Although the relative abundance of the Desulfovibrionaceae family (and of the Prevotellaceae family) is low compared to other bacterial families, the presence of the OTU for this family could only be found in the SOD1 mice (not detected in wild-type mice). This could suggest Desulfovibrionaceae playing a potential role in the physiopathology of SOD1 mice.

Study on Correlation in SOD1 Mice Treated or Untreated with the Low Dose of C1 (Combined with C2) for 10 Weeks

The RCCA carried out in the SOD1 control mice and those treated with the low dose of PL made it possible to demonstrate a strong positive correlation between the Lachnospiraceae family and the distance traveled by the mice. The more abundant this family, the greater the distance traveled by the mice. Given that one of the main effects of a composition according to the invention on the intestinal microbiota of the SOD1 mice is to increase the abundance of Lachnospiraceae species, the observed correlation suggests this bacterial family playing a role in the recovery of the ability of the SOD1 mice to travel a distance close to that traveled by the wild-type mice (FIG. 6).

The demonstration of a negative correlation between Desulfovibrionaceae and the severity of symptoms in the SOD1 mice suggests that species from this family, which produce sulfides, may be involved in physiopathology, as has been observed in other pathologies such as irritable bowel syndrome or chronic inflammatory bowel diseases (Crohn's disease, hemorrhagic rectocolitis). In parallel, the lower abundance of species from the Lachnospiraceae family in the SOD1 mice could lead to a decrease in the concentration of intracolic butyrate, since a large proportion of the species from Lachnospiraceae produce this metabolite with demonstrated effects on health.

The administration of a composition according to the invention at a low dose for 10 weeks is accompanied by changes in the composition of the microbiota of the SOD1 mice, resulting, in particular, in an increase in the abundance of Lachnospiraceae and the disappearance of Desulfovibionaceae. This correlates with a significant improvement in the ability of the mice to travel a distance equivalent to that of the wild-type mice. The administration of a composition according to the invention therefore partly rebalances the altered microbiota of the SOD1 mice, bringing about metabolic changes, and decreases the severity of certain symptoms in the SOD1 mice, or even restores them to a normal phenotype.

1. A composition comprising at least:
lactic acid, and/or a salt and/or ester and/or anhydride of lactic acid,
butyric acid, and/or a salt and/or ester and/or anhydride of butyric acid
propionic acid, and/or a salt and/or ester and/or anhydride of propionic acid,
for use in humans and animals for the prevention and/or treatment of pathological dysbiosis of the intestinal microbiota.

2. The composition for use according to the preceding claim, said prevention and/or said treatment being characterized by an increase in the proportion of butyrate-producing bacteria and/or a decrease in the proportion of sulfide-producing bacteria, in the intestinal microbiota.

3. The composition for use according to claim 1,
characterized in that the pathological dysbiosis of the intestinal microbiota is characterized by an excess of sulfide-producing bacteria and a deficiency of butyrate-producing bacteria.

4. The composition for use according to claim 3,
characterized in that the pathological dysbiosis of the intestinal microbiota is also characterized by an increase in intestinal inflammation.

5. The composition for use according to claim 1, in the prevention and/or treatment of at least one neurodegenerative disease and/or an intestinal disease associated with pathological dysbiosis of the intestinal microbiota.

6. The composition for use according to claim 5, characterized in that the neurodegenerative disease is selected from Lou Gehrig's disease, amyotrophic lateral sclerosis, multiple sclerosis, Parkinson's disease, and Alzheimer's disease.

7. The composition for use according to claim 5, characterized in that the intestinal disease is selected from among Crohn's disease, chronic inflammatory bowel diseases, hemorrhagic rectocolitis, irritable bowel syndrome, ulcerative colitis, rheumatoid arthritis and gluten intolerance.

8. The composition for use according to claim 1, as a drug or dietary supplement in humans or animals.

9. The composition for use according to claim 2, characterized in that the sulfide-producing bacteria belong to the Desulfovibrionaceae family and the butyrate-producing bacteria belong to the Lachnospiraceae family.

10. The composition for use according to claim 8, characterized in that bacteria belonging to the Desulfovibrionaceae family represent less than 0.01% of the total bacteria present in the intestinal microbiota.

11. The composition for use according to claim 1, characterized in that the composition comprises at least one polymer selected from among polylysine, polyethylene glycol, polyornithine, polyarginine and polyhistidine.

12. The composition for use according to claim 1, characterized in that at least one of the molecules of the composition selected from among lactic acid, butyric acid, propionic acid, salts of these acids, esters of these acids, and anhydrides of these acids, is covalently conjugated to at least one molecule of a polymer selected from among polylysine, polyethylene glycol, polyornithine, polyarginine and polyhistidine.

13. The composition for its use according to claim 1, characterized in that composition further comprises at least one molecule selected from:
oleic acid,
palmitic acid,
lauric acid,
linoleic acid,
azelaic acid,
farnesyl cysteine,
palmitoleic acid,
cholesterol,
thioctic acid,
myristic acid,
orotic acid,
pyruvic acid
acetic acid, and combinations thereof,
said molecule(s) potentially being in the form of a salt and/or an ester and/or an anhydride of one or more of these molecules.

14. The composition for use according to claim 1, characterized in that the composition comprises at least the following molecules:
oleic acid,
palmitic acid,
lauric acid,
linoleic acid,
azelaic acid,
farnesyl cysteine,
palmitoleic acid,
cholesterol,
thioctic acid,
myristic acid,
orotic acid,
pyruvic acid
acetic acid, and combinations thereof,
and/or a salt and/or an ester and/or an anhydride of one or more of these molecules.

15. The composition for use according to claim 1, characterized in that at least one of the molecules of the composition selected from among oleic acid, palmitic acid, lauric acid, linoleic acid, azelaic acid, palmitoleic acid, thioctic acid, myristic acid, orotic acid, acetic acid, butyric acid, lactic acid, propionic acid, salts of these acids, esters of these acids and anhydrides of these acids, is covalently conjugated to at least one molecule of a polymer selected from among polylysine, polyethylene glycol, polyornithine, polyarginine and polyhistidine.

16. The composition according to claim 12, characterized in that it comprises micelles wherein at least farnesyl cysteine and/or cholesterol and/or an ester of these molecules are encapsulated.

17. The composition according to claim 16, characterized in that at least one of the micelles is formed by amphiphilic conjugates each consisting of at least one hydrophobic molecule covalently conjugated to a molecule of a polymer selected from among polylysine, polyethylene glycol, polyornithine, polyarginine and polyhistidine.

18. The composition according to claim 17, characterized in that at least one micelle is formed by amphiphilic conjugates each consisting of at least one molecule selected from among oleic acid, palmitic acid, lauric acid, linoleic acid, palmitoleic acid, myristic acid, salts, esters and anhydrides of these fatty acids, covalently conjugated to a molecule of a polymer selected from among polylysine, polyethylene glycol, polyornithine, polyarginine and polyhistidine.

19. The composition for use according to claim 1, the composition being characterized in that at least one of the molecules is covalently conjugated to at least one polymer.

20. The composition for use according to claim 1, the composition being characterized in that it comprises at least:
A. the following conjugates, each conjugate consisting of a molecule covalently bonded to a polylysine:
one or more oleyl-poly-L-lysine conjugates
one or more palmitic-poly-L-lysine conjugates
one or more lauryl-poly-L-lysine conjugates
one or more azelayl-poly-L-lysine conjugates
one or more palmitoleyl-poly-L-lysine conjugates
one or more thioctyl-poly-L-lysine conjugates
one or more myristyl-poly-L-lysine conjugates
one or more orotyl-poly-L-lysine conjugates
one or more acetate-poly-L-lysine conjugates
one or more buyrate-poly-L-lysine conjugates
one or more lactate-poly-L-lysine conjugates
one or more propionate-poly-L-lysine conjugates,
one or more linoleyl-poly-L-lysine conjugates, and
B. farnesyl cysteine and cholesterol, and/or an ester of these molecules, encapsulated in micelles.

21. The composition for use according to claim 20, the composition being characterized in that farnesyl cysteine and cholesterol, and/or an ester of these molecules, are encapsulated in micelles formed by one or more of the conjugates from list A.

22. The composition for use according to claim 20, the composition being characterized in that the poly-L-lysine is replaced with another polylysine or with polyethylene glycol, a poly-L-ornithine, a poly-L-arginine or a poly-L-histidine.

23. The composition for use according to claim 1, characterized in that the composition comprises at least one pharmaceutically acceptable excipient.

24. The composition for use according to claim 1, characterized in that the composition is in liquid form or in solid form.
